(19) United States
(12) Patent Application Publication
(10) Pub. No.: US 2007/0225782 A1
(43) Pub. Date: Sep. 27, 2007

(54) Body cooling device

(76) Inventor: John Taylor, Manteca, CA (US)

Correspondence Address: ADVANTIA LAW GROUP, 9035 SOUTH 1300 EAST, SUITE 200, SANDY, UT, 84094, US

(21) Appl. No.: 11/228,931

(22) Filed: Sep. 15, 2005

(51) Int. Cl. A61F 7/00

(52) U.S. Cl. 607/108; 607/112

(57) ABSTRACT

There is a device for cooling a portion of a body. The device includes a water permeable chamber defining a cavity, water absorbent particles, and chambers or an obstacle member disposed between particles. The chambers are configured to be sufficiently small to prevent pooling of particles. The particles have a thermal conductivity constant less than that of ice. Further, there are eyeglass coupling members extending from ends of the device.

### BACKGROUND OF THE INVENTION

1. Field of the Invention

The present invention relates to cooling devices, specifically to devices for cooling a body.

2. Description of the Related Art

The skin is the largest organ in the human body. It protects the body from exposure to harmful rays, chemicals, and organisms. Another important function of the skin is temperature regulation. Skin temperature depends on air temperature and time spent in an environment. Weather factors such as wind chill and humidity can cause changes in skin temperature. The normal temperature of skin is about 33 degrees Centigrade, or 91 degrees Fahrenheit. However, there are many circumstances when it becomes more difficult for the skin to maintain proper internal temperatures, including but not limited to fevers and heat exposure. Also, such factors may cause discomfort to a person. Therefore, devices have been created to aid in maintaining a low skin temperature, thereby assisting the skin in combating high temperatures and/or comforting users.

Several methods of cooling skin and/or a body have developed. In extreme circumstances taking ice baths or bathing in cool water may be appropriate. People often spray water, apply wet cloth, soak clothing with water, expose themselves to moving air such as from a fan, etc. However, each of these methods/devices includes difficulties and/or inefficiencies such as difficult use, uncomfortable cooling, in-portability, weak cooling effect, damage to clothing, and messiness.

There are devices intended to be used to cool parts of bodies that attempt to cool bodies in more convenient ways. For example, U.S. Pat. No. 5,755,110 to Silvas discloses a cooling vest having a plurality of elongated pocket partitions formed on front side and backside vest portions for containing beads of polyacrylamide material that absorb a liquid, such as water, to form a gel that may be chilled, or chilled to near freezing, to provide a cooling effect on the upper torso of a human wearer. The cooling effect is facilitated by non-impervious properties of a double layer fabric used in construction of the vest that permit evaporation. The front side portions of the vest also provide surface area upon which to add fashion appeal ornate designs, or on which utility pockets are formed. The vest is further provided with a collar member having a partition for containing polymer material. The upper shoulder blade area of the backside of the vest is divided from the lower lumbar area by a stitching pattern that ornate the backside of the vests and that separates the upper and lower polymer containing partitions. The shoulder blade pocket partitions are thinner because these partitions contain less polymer material than the lower partitions that define the lumbar region of the vest. The lower lumbar area of the vest is formed to contain more of the polymer material primarily to provide an additional lumbar support feature. The underarm regions of the vest include band pocket partitions to especially provide cooling to that part of a user's body. An alternate vest embodiment includes mesh fabric strips adjacent each pocket partition.

Further, U.S. Patent Application No. 2004/0226077 to Toth discloses systems and methods for providing an article associated with headgear that keeps the wearer's head both cool and cushioned from the interior harness of the headgear. The article is a system, liner or pad that is inserted into the headgear or coupled to the headgear harness for cooling and comfort, and includes a water absorbent polymer or other material contained within non-impervious pocket partitions attached to a concave disk of mesh fabric. The mesh fabric provides ventilation portions, which allow for the free flow of air and breathing of the wearer's scalp to facilitate the natural evaporation of perspiration. The article provides a cushion to the wearer's scalp from the abrasive effect of the webbing of the headgear harness. As the pocket partitions swell due to absorption of water by the polymer material, the pockets function as gel-like cushions or pads that protect the wearer's scalp. The article is held in place with small straps, Velcro tabs, or other retention devices.

Still further, U.S. Patent Application No. 2002/0076533 to Cacered et al. discloses a cooling article comprising a polymer absorbent enclosed within a bag delimited by a collapsible envelope having non-watertight walls, wherein said polymer absorbent is under the form of particles each of which comprises a core of less cross-linked polymer sequences more active in retaining absorbed water and a shell of more cross-linked polymer sequences apt to retard diffusion of water from a particle to another during desorption of absorbed water.

Problems with prior art cooling devices include, but are not limited to, a slimy feel, too rapid heat transfer, pooling of water absorbent particles, inconsistencies between function and/or configuration of the product between successive hydration/dehydration cycles, improper fitting to body parts, toxic and/or hazardous components, non-biodegradable components, expensive components, and only single function use.

Accordingly, there exists a need for a body cooling device that solves one or more of the problems herein described or that may come to the attention of one skilled in the art after becoming familiar with this specification.

### SUMMARY OF THE INVENTION

The present invention has been developed in response to the present state of the art, and in particular, in response to the problems and needs in the art that have not yet been fully solved by currently available body cooling devices. Accordingly, the present invention has been developed to provide a body cooling device.

In one embodiment there is a device for cooling a portion of a body, including a plurality of chambers each defining a cavity having a cavity volume and a water absorbent particle disposed within each cavity. It may be that the first cavity volume is less than about twelve times the volume of the associated fully hydrated water absorbent particle. There may be further included an adhesive layer coupled to at least one of the plurality of chambers and/or a release layer coupled to the adhesive layer.

In another embodiment, there may be a plurality of water absorbent particles disposed within the first cavity. It may be that the first cavity volume is between about 0.5 or about 0.7 times to about 0.8 or to about 0.95 times the total volume of the plurality of fully hydrated water absorbent particles. It may be that each cavity volume is less than about ten times the volume of each associated fully hydrated water absorbent particle. Also, it may be that the water absorbent particle comprises a cationic super absorbent polymer. There may also be included a planar member defined by the plurality of chambers, wherein the plurality of chambers are interconnected.

Still more, it may be that the water absorbent particle comprises a thermal conductivity constant when hydrated less than about two-thirds or less than about half or less than about one quarter of that of water of the same temperature. There may be a water absorbent particle that may comprise a thermal conductivity constant when hydrated and chilled to near freezing of less than about one half, less than about one third, or less than about one quarter of that of ice.

It may be that the plurality of chambers each further comprise a capacity no greater than about 23, 27, or 32 cubic centimeters and a longest dimension no greater than about 7, 9, or 10 centimeters. Also, it may be that each of the plurality of chambers includes first, second, and third engorged lengths, wherein the first, second, and third engorged lengths are mutually orthogonal, the third engorged length is about the largest internal length possible to select, and the first and second engorged lengths are smaller than about a diameter of an associated fully hydrated water absorbent particle. More, it may be that the water absorbent particle comprises a thermal conductivity constant sufficiently small to prevent injurious freezing when hydrated, chilled to near freezing, and applied for any length of time to a body of a person.

In another embodiment there may be a device for cooling a portion of a body. The device may include a water permeable chamber defining a cavity, first and second water absorbent particles each disposed within the cavity, and/or an obstacle member disposed within the cavity between the first and second water absorbent particles, and/or configured to resist motion of the first water absorbent particle towards the second water absorbent particle. It may be that the obstacle member comprises being coupled to an interior surface of the chamber. Further, it may be that the obstacle member comprises a fiber having first and second ends coupled to an interior of the chamber. More, it may be that the obstacle member comprises a fibrous layer disposed within the cavity and coupled to the chamber by offset quilting.

Additionally, the obstacle member may be not coupled to the water permeable chamber. Also, the water permeable chamber may further comprise first, second, and third engorged lengths, wherein the first, second, and third engorged lengths are mutually orthogonal, the third engorged length is about the largest internal length possible to select, and the first and second engorged lengths are smaller than about a diameter of an associated fully hydrated water absorbent particle. It may also be that the obstacle member comprises a water absorbent particle having a diameter, when hydrated, greater than each of the first and second engorged lengths.

In one embodiment there may be a device for cooling a portion of a body. The device may include a planar cooling member, having a plurality of interconnected chambers each defining a cavity having a cavity volume and first and second ends. There may also be a water absorbent particle disposed within each cavity. More, there may be first and second linear eyeglass coupling members extending from the first and second ends respectively and each configured to couple to eyeglasses.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates a side perspective view of a body cooling device according to one embodiment of the invention;

FIGS. 2 and 3 illustrate cross-sectional views of a body cooling device in hydrated and non-hydrated modes respectively;

FIGS. 4 and 5 illustrate cross-sectional views of a hydrated and an non-hydrated SAP particle according to one embodiment of the invention;

FIG. 6 illustrates a side perspective view of a body cooling device according to one embodiment of the invention;

FIG. 7 illustrates a side perspective view of a pair of body cooling devices according to one embodiment of the invention;

FIG. 8 illustrates a side perspective view of a process of making a body cooling device according to one embodiment of the invention;

FIG. 9 illustrates a top plan view of a body cooling device according to one embodiment of the invention; and

FIG. 10 illustrates perspective views of a pair of body cooling devices according to one embodiment of the invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

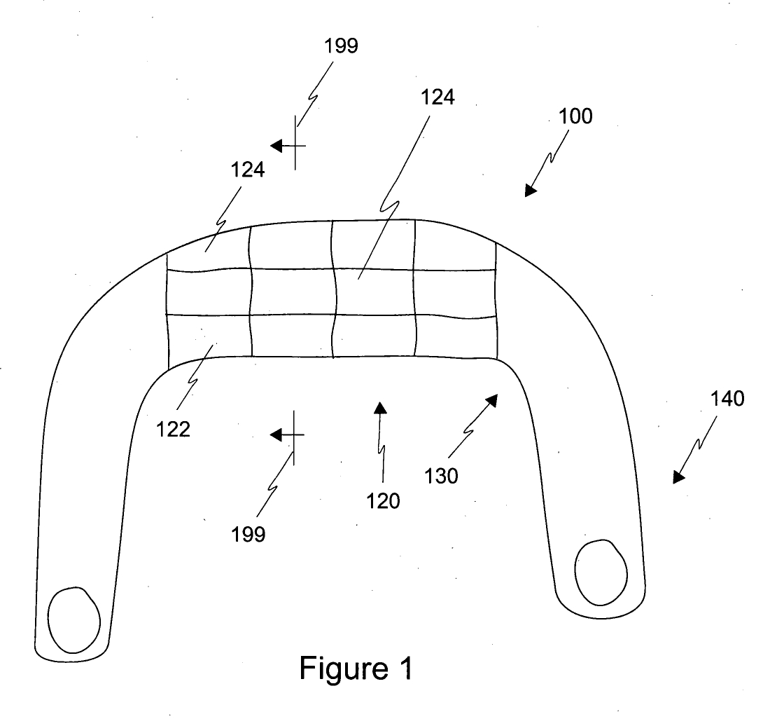

FIG. 1 illustrates a side perspective view of a body cooling device 100 according to one embodiment of the invention. There is shown an application member 120, a pair of extending members 130 and a pair of coupling members 140. The body cooling device 100 is configured to couple to a body and participate in heat transfer therewith. The body cooling device 100 may be used to cool, heat, and/or buffer (maintain) a body temperature.

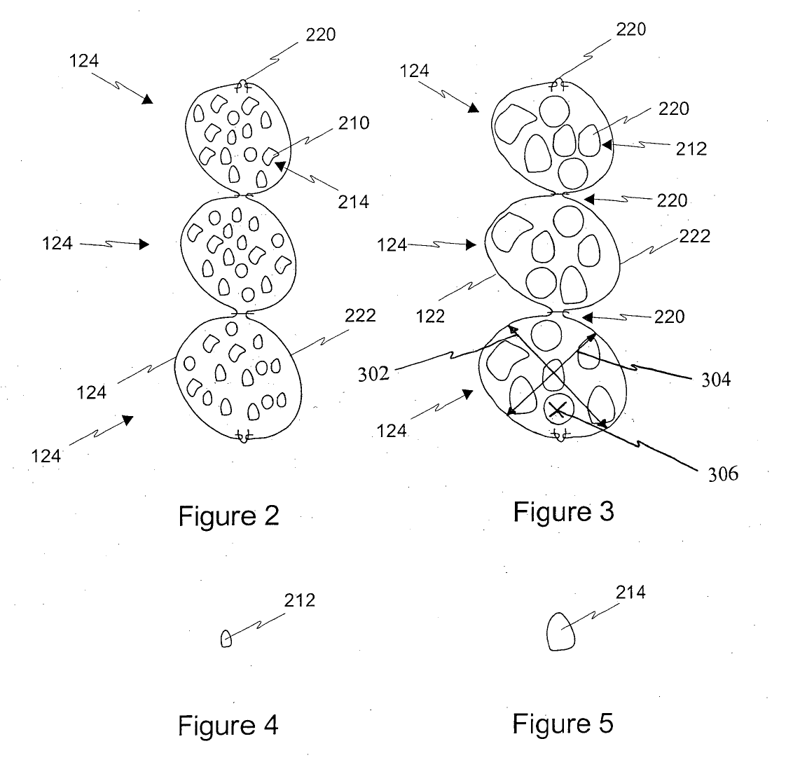

The application member includes a first surface 122 coupled to a second surface (not shown, see FIGS. 2 and 3). The first 122 and/or the second surface may be substantially water permeable, flexible, and/or elastic. Also, the application member 120 is portioned into a plurality of chambers 124. The plurality of chambers 124 preferably include water absorbent materials. Preferably the water absorbent materials include super absorbent polymers (SAP). The plurality of chambers 124 may be formed by stitching together the first 122 and second surfaces along lines configured to produce chambers 124. In particular, there may be intersecting coupling lines coupling the first and second surfaces thereby forming chambers. Examples of coupling lines include but are not limited to stitching, gluing, welding, pinching, clasping, and any other methods and structures known in the art for coupling a pair of planar members together at a line or point.

In another example, there may be offset quilting. There may be a fibrous material disposed between one or more pairs of surfaces. The fibrous material may provide an anchoring layer for coupling such as but not limited to stitching. The fibrous material may also tend to hold water absorbent materials in a matrix, thereby resisting pooling. There may be one or more coupling lines coupling a first surface to a fibrous material and one or more coupling lines coupling a second surface to the fibrous material. Such coupling lines may be located and configured to fix a location of the fibrous material with respect to the first and second surfaces without ever fully pinching both the first and second surfaces together.

Though generally rectangular chambers 124 are shown, it is envisioned that any shape of chamber may thereby be constructed, including but not limited to circles, squares, ellipses, irregular shapes, character shapes, letters, and trademarks. Also, although the chambers 124 shown are generally identical, there may be a plurality of shapes and/or sizes of chambers 124 included in a single body cooling device 100.

The extending members 130 and the coupling members 140 may be configured to permit coupling of the body cooling device 100 about a body. In particular, it may be desired to couple a body cooling device 100 about an extremity, such as but not limited to a hand or leg of a human. An extending member 130 may permit placement of a coupling member 140 in a location whereat the coupling member 140 may couple to another object, including but not limited to another coupling member 140 and/or clothing of an individual. Preferably an extending member is made of a flexible material that may be elastic, thereby permitting the extending member to be moved, pulled, stretched twisted, etc. in modes facilitating attachment of the body cooling device 100 to a body. A coupling member 140 may be of any variety known in the art, including, but not limited to hook and loop, snaps, buttons, zippers, ties, wraps, plastically deformable portions, holes, clips, pins, hooks, interlocking members, and buckles.

In one example, a chamber may include an obstructing member such as but not limited to a fibrous interior, a divider, an internal protrusion, a free divider, a stitch, a glue, and a planar member having a comparatively high coefficient of friction disposed substantially parallel to the plane of the flattened chamber. There may be a plurality of fibers coupled to an interior portion of a chamber. Wherein a non-hydrated SAP is typically rigid, granular, and odd-shaped, a non-hydrated particle may catch on an obstructing member, such as one or more fibers, and not pool in a portion of a chamber, thereby keeping a more even distribution of particles throughout a chamber.

In one embodiment there is a device for cooling a portion of a body, including a plurality of chambers each defining a cavity having a cavity volume and a water absorbent particle disposed within each cavity. It may be that the first cavity volume is less than about twelve times the volume of the associated fully hydrated water absorbent particle. There may be further included an adhesive layer coupled to at least one of the plurality of chambers and/or a release layer coupled to the adhesive layer.

In another embodiment, there may be a plurality of water absorbent particles disposed within the first cavity. It may be that the first cavity volume is between about 0.5 or about 0.7 times to about 0.8 or to about 0.95 times the total volume of the plurality of fully hydrated water absorbent particles. Advantageously, such relative volumes prevent pooling of particles that may otherwise occur with larger volumes, thereby facilitating a better distribution of heat exchange capacity while still enabling substantial absorption. It may be that each cavity volume is less than about ten times the volume of each associated fully hydrated water absorbent particle. Also, it may be that the water absorbent particle comprises a cationic super absorbent polymer. There may also be included a planar member defined by the plurality of chambers, wherein the plurality of chambers are interconnected. Such may advantageously form a planar member having a portioned bed having a relatively high thermal capacity.

Still more, it may be that the water absorbent particle comprises a thermal conductivity constant when hydrated less than about two-thirds or less than about half or less than about one quarter of that of water of the same temperature. There may be a water absorbent particle that may comprise a thermal conductivity constant when hydrated and chilled to near freezing of less than about one half, less than about one third, or less than about one quarter of that of ice. Advantageously, such prevents cold “burns” associated with direct application of ice to a human body. Further, such provides a more comfortable transfer of heat between a human body and a device. More, it may be that the water absorbent particle comprises a thermal conductivity constant sufficiently small to prevent injurious freezing when hydrated, chilled to near freezing, and applied for any length of time to a body of a person.

It may be that the plurality of chambers each further comprise a capacity no greater than about 23, 27, or 32 cubic centimeters and a longest dimension no greater than about 7, 9, or 10 centimeters. Also, it may be that each of the plurality of chambers includes first, second, and third engorged lengths, wherein the first, second, and third engorged lengths are mutually orthogonal, the third engorged length is about the largest internal length possible to select, and the first and second engorged lengths are smaller than about a diameter of an associated fully hydrated water absorbent particle.

In another embodiment there may be a device for cooling a portion of a body. The device may include a water permeable chamber defining a cavity, first and second water absorbent particles each disposed within the cavity, and/or an obstacle member disposed within the cavity between the first and second water absorbent particles, and/or configured to resist motion of the first water absorbent particle towards the second water absorbent particle. It may be that the obstacle member comprises being coupled to an interior surface of the chamber. Further, it may be that the obstacle member comprises a fiber having first and second ends coupled to an interior of the chamber. More, it may be that the obstacle member comprises a fibrous layer disposed within the cavity and coupled to the chamber by offset quilting.

Additionally, the obstacle member may be not coupled to the water permeable chamber. Also, the water permeable chamber may further comprise first, second, and third engorged lengths, wherein the first, second, and third engorged lengths are mutually orthogonal, the third engorged length is about the largest internal length possible to select, and the first and second engorged lengths are smaller than about a diameter of an associated fully hydrated water absorbent particle. It may also be that the obstacle member comprise a water absorbent particle having a diameter, when hydrated, greater than each of the first and second engorged lengths.

FIGS. 2 and 3 illustrate cross-sectional views of a body cooling device in non-hydrated and hydrated modes respectively. The cross-sectional view is taken through lines 199 in FIG. 1. There may be a chamber 124 or a plurality of chambers 124 such as the three chambers illustrated. A chamber 124 may contain one or more water absorbent particles 210 such as but not limited to SAP particles. When a water absorbent particle 210 is non-hydrated (when the particle does not contain a substantial amount of water) 212 it is smaller than when the water absorbent particle 210 is hydrated 214 (when the particle contains a substantial amount of water). A chamber 124 may be configured to expand as a water absorbent particle 210 expands during saturation. A chamber 124 may be elastic. A chamber 124 may be of a flexible material that may lay substantially flat when a water absorbent particle 210 contained within is non-hydrated, but may engorge, expand, and/or inflate upon saturation of the water absorbent particle.

There may be a plurality of chambers 124 defined by stitching 220 coupling a pair of sheets of material 122 and 222 at desired points. Thereby a pair of sheets of material 122 and 222 may be partitioned into a plurality of chambers 124 wherein a plurality of water absorbent particles 210 may be contained. Thereby the plurality of water absorbent particles 210 may be prevented from all collecting at a single portion of the device.

There exist a variety of SAP materials. In particular, SAP particles are often used in baby diapers (and other personal sanitation products) to rapidly absorb liquids. While the SAP materials used in baby diapers typically rapidly absorb liquids, there are substantial problems when using such SAP materials for body cooling devices. In particular, baby diaper SAP materials have a slimy feel, may bleed through fabric, and typically have a high rate of heat transfer. A high rate of heat transfer may be undesirable wherein a body cooling device may be chilled to near freezing and applied directly to the skin of a user. In such a case a body cooling device using baby diaper SAP material may cause discomfort and/or injury by too rapidly cooling the skin to too low of a temperature. Further, a body cooling device using baby diaper SAP material may too quickly transfer heat and thereby not provide a consistent cooling effect because such a body cooling device may quickly approach room/body temperature.

Preferably a body cooling device will use a SAP material that transfers heat at a sufficiently low rate to permit direct or near direct application of the materials to a portion of skin without endangering the skin, even when the SAP material is chilled to near freezing. One particular SAP material shown to have properties superior to those of the baby diaper SAP materials is Cationic Polyacrylamide, or superabsorbent polyacrylatelamide, such as those sold under the trade name Aquarocks and supplied by W.A. Industries, Inc. having a place of business at 700 W. Van Buren St. Suite 1405 in Chicago Ill. Superabsorbent polyacrylatelamide is biodegradable, breaking down into water, carbon-dioxide, and nitrogen after approximately 7-9 years. Examples of properties of such materials are illustrated in FIGS. 11 and 12. Further, superabsorbent polyacrylatelamide is non-hazardous and not a significant ecological threat.

FIGS. 4 and 5 illustrate cross-sectional views of a non-hydrated and a hydrated SAP particle according to one embodiment of the invention. In particular, there is shown an non-hydrated SAP particle 212 and a hydrated SAP particle 214. The hydrated SAP particle 214 is significantly larger than the non-hydrated SAP particle 212. Further, when hydrated, an SAP particle loses rigidity and hardness.

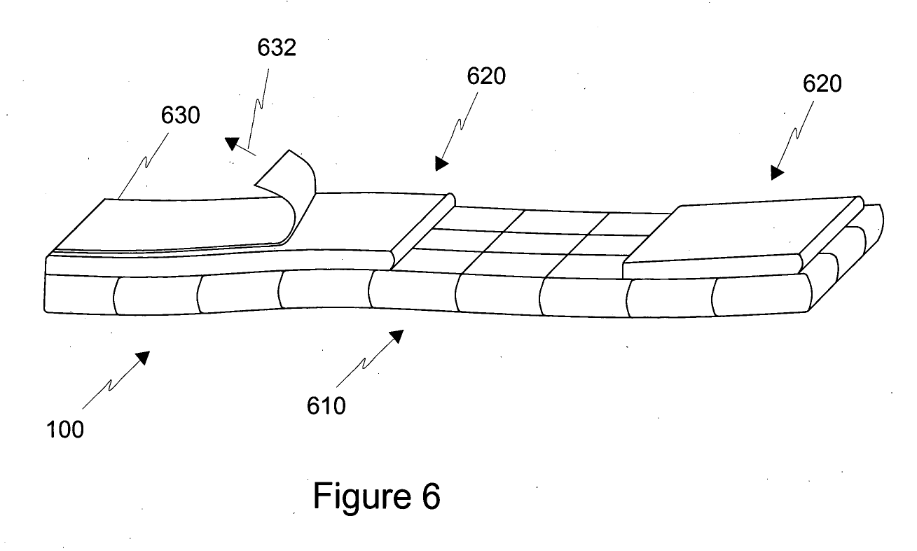

FIG. 6 illustrates a side perspective view of a body cooling device according to one embodiment of the invention. There is shown a body cooling device 100 including a chambered portion 610 an adhesive layer 620 and a release layer 630. The chambered portion 610 may include water absorbent materials such as SAP particles. Preferably the adhesive layer 620 includes a pressure sensitive adhesive having adhesive properties not strongly inhibited by exposure to liquids such as water. Also, it is preferred that the adhesive layer not be a thermal insulator. The release layer 630 is configured to couple to the adhesive layer 620, protecting the same until the device is to be used.

In operation, the body cooling device 100 is prepared by soaking in water of a desired temperature for a determined period of time. Preferably the body cooling device 100 is permitted to soak in water at least until a majority of the water absorbent material is fully hydrated. Then one or more release layers 630 may be removed, as shown by the motion indicated by arrow 632, from the body cooling device 100 and the body cooling device may be applied to a portion of a body of a user by pressing the adhesive against a surface such as skin of the user about a portion of the body intended to be cooled. Preferably, a body cooling device is re-usable and/or disposable.

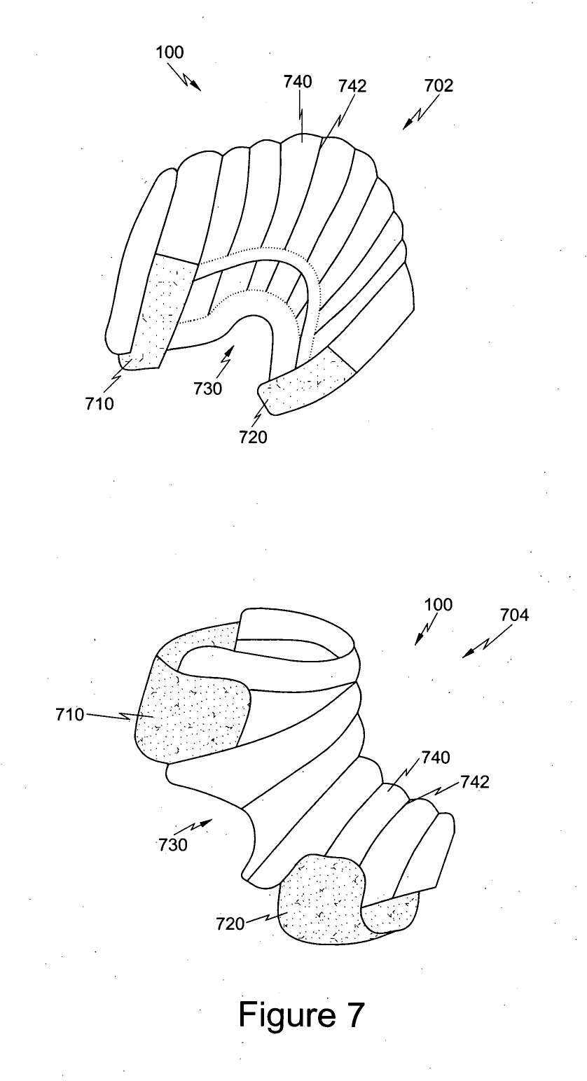

FIG. 7 illustrates a sides perspective views of body cooling devices according to one embodiment of the invention. There is shown a pair of body cooling devices 100, in particular an elbow and/or knee compress 702 and an ankle compress 704, each configured to couple to a portion of a body that bends, such as a knee or elbow. There is shown a first strap 710 a second strap 720 and a body conforming member 730. The first and second straps 710 and 720 are configured to couple the body cooling device 100 to a body and prevent detachment thereof. The body conforming member 730 is configured to approximate a shape of a body portion, such as a knee, ankle, elbow, etc. Further, the body conforming member 730 comprises materials such as fabric, elastic, plastic, gel, hydrated SAP, etc. that may change shape and/or size under pressure, thereby permitting the body conforming member 730 to further adapt to conform to a shape of a body portion to which it may be attached.

There are illustrated a plurality of chambers 740 defined by barriers 742 that may be stitching, sealed portions, coupled portions, etc. The plurality of chambers 740 include SAP material, preferably in an amount sufficient to engorge and/or fill each chamber when the SAP materials therein are hydrated. In one example, a chamber includes sufficient SAP material such the chamber has a maximum volume less than or equal to a total volume of the SAP material when the SAP material is fully hydrated. In another example, the chamber has a maximum volume less than about 90% of a maximum volume of the associated SAP material. In another example, each of the plurality of chambers 740 have a maximum volume relating to a total volume of associated SAP material as described above.

In one example a chamber 740 may be substantially longitudinal as shown in the figures and may be aligned substantially parallel to an axis of rotation of a body part when the body cooling device 100 is coupled to the body. In one example, a chamber 740 may substantially encircle a body part when the body cooling device 100 is coupled to the body part.

In operation, a body cooling device 100 may be approximately shaped to include a cavity resembling a shape of a body portion intended to be enclosed and/or ensconced therein. The body cooling device may be hydrated and cooled, perhaps even near freezing. The body cooling device 100 may be stored such that the body cooling device 100 remains substantially hydrated and/or at a desired temperature. When needed, such as at the occurrence of an injury during a sporting event, the body cooling device 100 may be withdrawn from storage (for example, taken from a cooler including ice water) and then applied to a body portion in need of cooling. Preferably, a body cooling device 100 is not frozen, as freezing SAP material may cause the SAP material to degenerate. Also, wherein materials are used that have a heat transfer rate within a desired range, there may be no need for any additional layers, such as towels, to protect the body portion from too much cold.

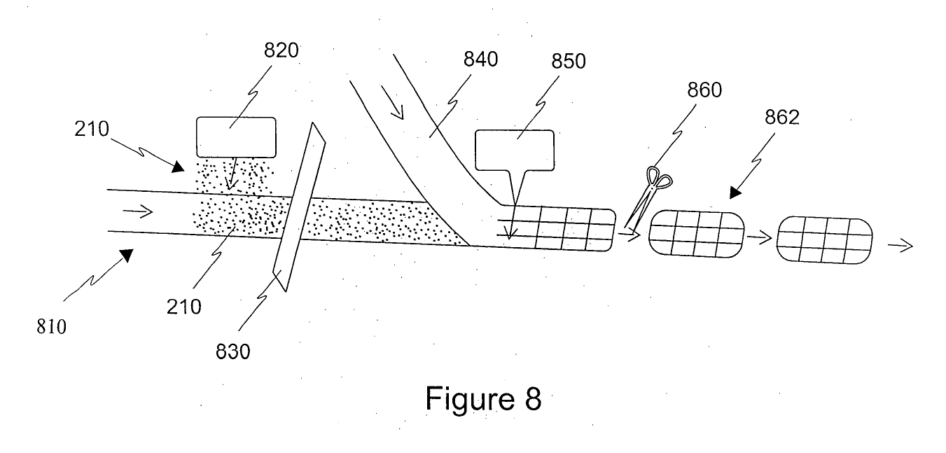

FIG. 8 illustrates a side perspective view of a process of making a body cooling device according to one embodiment of the invention. There is shown a first containment layer 810 moving through an assembly process. There is a hopper 820 depositing a quantity of water absorbent particles 210 over the first containment layer 810. There is also a distribution member 830 configured to help maintain a proper distribution of particles 210 over the surface of the first containment layer 810. There is a second containment layer 840 covering the particles 210 and the first containment layer 810. There is a sewing machine 850 configured to sew the first containment layer 810 to the second containment layer 840. There is a cutting device 860 configured to cut through the first and second containment layers 810 and 840 thereby forming chambered members 124 for use in making body cooling devices.

In operation, there may be a substantially continuous manufacturing process. A first containment layer 810 may be moved towards the hopper 820. The first containment layer 810 is preferably a water permeable fabric. Water absorbent particles 210 from the hopper 820 are deposited over an upper surface of the first containment layer 810. Preferably the particles 210 are deposited according to a predefined pattern, evenly, or randomly. A distribution member 830 may be included that may be a ridge of material in a determined shape configured to help distribute the particles according to manufacturing needs. For example, a distribution member 830 may be a ridge disposed close to the first containment layer 810 and may therefore level off high particle distributions. A distribution member 830 may move according to a defined pattern or in response to indicia, such as information received from sensors.

A second containment layer 840 may be disposed above the first containment layer 810 and may be lowered to rest thereon. One or more sewing machines or devices 850 may couple the first and second containment layers 810 and 840. Preferably, the first and second containment layers 810 and 840 are coupled along a plurality of intersecting lines, thereby forming a plurality of chambers. Preferably, there is at least a single particle included in each chamber. One skilled in the art Would be able to determine an appropriate or ideal amount of water absorbent material to include in a chamber and could configure a manufacturing process to produce such within appropriate error parameters.

A cutting device 860 may cut portions of the first and second containment layers 810 and 840, preferably after chambers are formed therewith. Preferably, the cutting device 860 will cut the material into appropriately sized portions for use in body cooling devices. The cut portions 862 may continue to further processes.

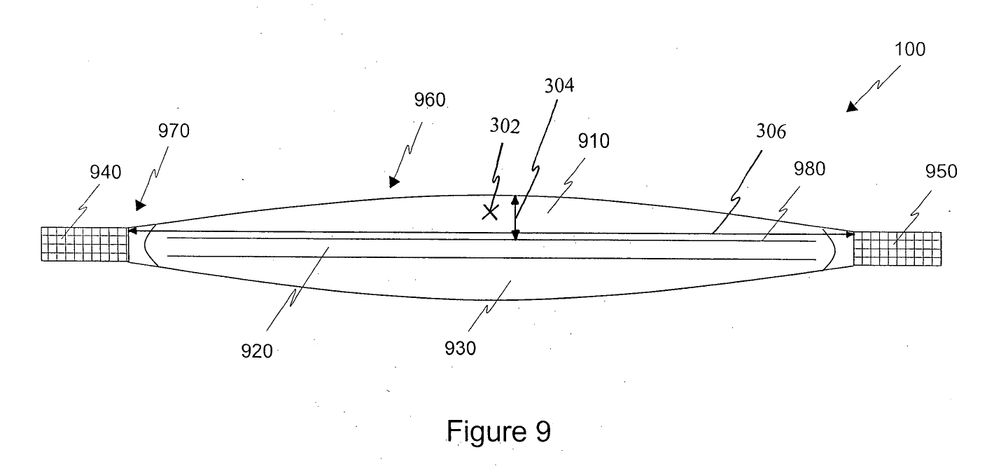

FIG. 9 illustrates a top plan view of a body cooling device according to one embodiment of the invention. There is illustrated a body cooling device 100 including first, second, and third chambers 910, 920, and 930, and first and second coupling portions 940 and 950. There is a central region 960 of the chambers 910, 920, and 930 that is wider than an end 970 of the body cooling device 100. Thereby the body cooling device may be adapted to more comfortably fit about the head of a person as well as reducing a total amount of material required to construct a body cooling device. Water absorbent materials are included in each of chambers 910, 920, and 930. The chambers 910, 920, and 930 are partitioned by coupling lines 980.

In operation, the body cooling device 100 may be submerged in a cool aqueous liquid for a time sufficient to hydrate the water absorbent materials to a desired degree. Then the body cooling device may be placed about a head or neck of a user with the central region being placed proximate the skin of the user at a region of the user desired to be cooled. The first and second coupling portions 940 and 950, preferably of hook and loop (but may be any coupling devices known in the art such as but not limited to buttons, snaps, and ties), may connect, thereby coupling the body cooling device about a person, facilitating keeping the body cooling device proximate a portion of skin intended to be cooled.

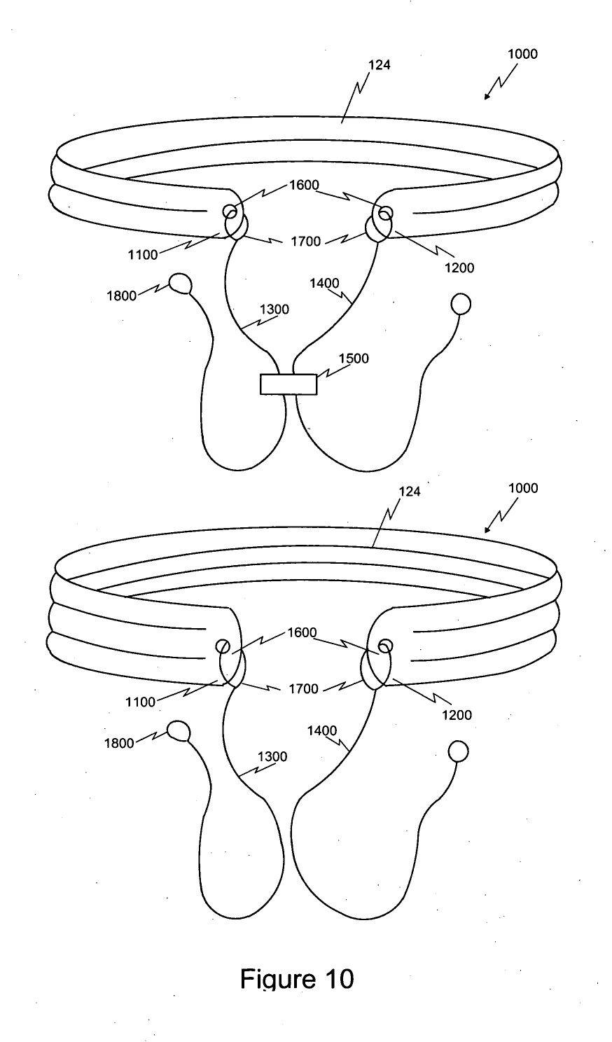

FIG. 10 illustrates perspective views of a pair of body cooling devices according to one embodiment of the invention. There is shown a first combined body cooling device/eyeglass holder 1000. There is shown a second combined body cooling device/eyeglass holder 1000. Both embodiments are configured for simultaneous utility of both aspects of each embodiment.

Included in the first combined body cooling device/eyeglass holder 1000 are a plurality of chambers 124, first and second ends 1100 and 1200, first and second linear members 1300 and 1400 and a locking member 1500. The plurality of chambers 124 are substantially as described in previously described embodiments. The first and second ends 1100 and 1200 include one or more connecting devices 1600 such as grommets for connecting to other connecting devices. In the illustrated example, there are grommets configured to couple to eyes 1700 formed at ends of each of the first and second linear members 1300 and 1400. Each of the first and second linear members 1300 and 1400 couples to an end 1100 or 1200 of the body cooling device 100 and extends therefrom. Also, each of the first and second linear members 1300 and 1400 includes a eyeglass coupling member 1800 at another end of the linear member, wherein the eyeglass coupling member is configured to couple the combined device to a set of eyeglasses, thereby enabling a user to simultaneously cool a head or a neck while being able to wear a set of eyeglasses held about the neck. An eyeglass coupling member may be any known in the art, such as but not limited to socks, loops, cinches, ties, snaps, etc. There is also shown a locking member 1500 configured to couple together each of the linear members 1300 and 1400. The locking member 1500 may be a sliding locking member, such as a slip lock, thereby enabling a user to alter a locking location and tightness. The locking member 1500 may be used to alter a body cooling device circumference, thereby enabling a user to alter a mode of fitting the device about a body part.

Included in the second combined body cooling device/eyeglass holder 1000 are a plurality of chambers 124, first and second ends 1100 and 1200, and first and second linear members 1300 and 1400. The plurality of chambers 124 are substantially as described in previously described embodiments. The first and second ends 1100 and 1200 include one or more connecting devices 1600 such as grommets for connecting to other connecting devices. In the illustrated example, there are grommets configured to couple to eyes 1700 formed at ends of each of the first and second linear members 1300 and 1400. Each of the first and second linear members 1300 and 1400 couples to an end 1100 or 1200 of the body cooling device 100 and extends therefrom. Also, each of the first and second linear members 1300 and 1400 includes a eyeglass coupling member 1800 at another end of the linear member, wherein the eyeglass coupling member is configured to couple the combined device to a set of eyeglasses, thereby enabling a user to simultaneously cool a head or a neck while being able to wear a set of eyeglasses held about the neck. One or more linear members 1300 and/or 1400 may be of a fine material thereby adding little or no significant weight to the total weight of the device. In use, the body cooling device may couple about a neck of the wearer, with the linear members crossing at a back of the neck of the wearer and then coupling to opposite ear portions of eyeglasses.

In order to demonstrate the practice of the present invention, the following example has been prepared. The example should not, however, be viewed as limiting the scope of the invention. The claims will serve to define the invention.

### Example According to One Embodiment of the Invention

A body cooling device including a plurality of chambers having superabsorbent polyacrylatelamide particles therein was hydrated in plain tap water for 20 min. Then ice was added to the water and measured a temperature of 42 degrees F. The body cooling device was soaked in the ice water for 10 minutes. When removed from the ice water the temperature of the cooler measured 48 degrees. The body cooling device was applied to a thigh of a person and measured the temperature between the skin and the body cooling device to be 68 degrees. After 15 min the temperature went up to 78 degrees. In 40 minutes the temperature did not rise over 80 degrees. Ambient room temperature was 78 degrees. After 1 hour in moving air the temperature remained at 78 to 80 degrees.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope. For example, but not by way of limitation, wherein a body cooling device is incorporated into an article of clothing, such is within the scope of the claims.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

1. A device for cooling a portion of a body, comprising:
a plurality of chambers each defining a cavity having a cavity volume; and
a water absorbent particle disposed within each cavity; wherein a first cavity volume is less than about twelve times the volume of the associated water absorbent particle when the associated water absorbent particle is fully hydrated.

2. (canceled)

3. The device of claim 1, further comprising:
an adhesive layer coupled to at least one of the plurality of chambers; and
a release layer coupled to the adhesive layer.

4. The device of claim 1, further comprising a plurality of water absorbent particles disposed within a first cavity, wherein the first cavity volume is between about 0.7 times to about 0.95 times the total volume of the plurality of fully hydrated water absorbent particles.

5. The device of claim 1, wherein each cavity volume is less than about ten times the volume of each associated fully hydrated water absorbent particle.

6. The device of claim 1, wherein the water absorbent particle comprises a cationic super absorbent polymer.

7. The device of claim 1, further comprising a planar member defined by the plurality of chambers, wherein the plurality of chambers are interconnected.

8. The device of claim 1, wherein the water absorbent particle comprises a thermal conductivity constant when hydrated less than about half of that of water of the same temperature.

9. The device of claim 1, wherein the water absorbent particle comprises a thermal conductivity constant when hydrated and chilled to near freezing of less than about one third of that of ice.

10. The device of claim 1, wherein the plurality of chambers each further comprise a capacity no greater than about 27 cubic centimeters and a longest dimension no greater than about 9 centimeters.

11. The device of claim 1, wherein each of the plurality of chambers includes:
first, second, and third engorged lengths, wherein the first, second, and third engorged lengths are mutually orthogonal, the third engorged length is about the largest internal length possible to select, and the first and second engorged lengths are smaller than about a diameter of an associated fully hydrated water absorbent particle.

12. The device of claim 1, wherein the water absorbent particle comprises a thermal conductivity constant sufficiently small to prevent injurious freezing when hydrated, chilled to near freezing, and applied for any length of time to a body of a person.

13. A device for cooling a portion of a body, comprising:
a water permeable chamber defining a cavity;
first and second water absorbent particles each disposed within the cavity; and
an obstacle member disposed within the cavity between the first and second water absorbent particles, and configured to resist motion of the first water absorbent particle towards the second water absorbent particle.

14. The device of claim 13, wherein the obstacle member comprises being coupled to an interior surface of the chamber.

15. The device of claim 13, wherein the obstacle member comprises a fiber having first and second ends coupled to an interior of the chamber.

16. The device of claim 13, wherein the obstacle member comprises a fibrous layer disposed within the cavity and coupled to the chamber by offset quilting.

17. The device of claim 13, wherein the obstacle member is not coupled to the water permeable chamber.

18. The device of claim 13, wherein the water permeable chamber further comprises first, second, and third engorged lengths, wherein the first, second, and third engorged lengths are mutually orthogonal, the third engorged length is about the largest internal length possible to select, and the first and second engorged lengths are smaller than about a diameter of an associated fully hydrated water absorbent particle.

19. The device of claim 18, wherein the obstacle member comprise a water absorbent particle having a diameter, when hydrated, greater than each of the first and second engorged lengths.

20. A device for cooling a portion of a body, comprising:
a planar cooling member, including:
a plurality of interconnected chambers each defining a cavity having a cavity volume; and
first and second ends;
a water absorbent particle disposed within each cavity; and
first and second linear eyeglass coupling members extending from the first and second ends respectively and each configured to couple to eyeglasses.

21. The device of claim 20, wherein the first and second ends each comprise a connecting device, and the first and second linear eyeglass coupling members each comprise an eye coupled to the connecting devices, respectively.
